(19) United States
(12) Patent Application Publication
(10) Pub. No.: US 2010/0151441 A1
(43) Pub. Date: Jun. 17, 2010

(54) Human Cytomegalovirus Latency Promoting Genes, Related Virus Variants and Methods of Use

(75) Inventors: Thomas E Shenk, Princeton, NJ (US); Felicia D. Goodrum, Tucson, AZ (US)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: THE TRUSTEES OF PRINCETON UNIVERSITY

(21) Appl. No.: 11/992,337

(22) PCT Filed: Sep. 22, 2006
(86) PCT No.: PCT/US2006/037058

§ 371 Date: Nov. 25, 2009

Related U.S. Application Data
(60) Provisional application No. 60/719,858, filed on Sep. 23, 2005.

(51) Int. Cl. C12Q 1/70; C12N 7/00; C12Q 1/68

(52) U.S. Cl. 435/5; 435/235.1; 435/6

(57) ABSTRACT

Latency promoting genomic sequences from human cytomegalovirus (HCMV) and virus variants lacking function of one or more of the latency promoting genes are disclosed. Also disclosed are methods of using the altered viruses and latency promoting genes and their gene products for the production of vaccines and for identifying antiviral compounds.

### CROSS-REFERENCE TO RELATED APPLICATIONS

This claims benefit of U.S. Provisional Patent Application No. 60/719,858, filed Sep. 23, 2005, which is incorporated by reference herein, in its entirety.

Pursuant to 35 U.S.C. §202 (c), it is acknowledged that the United States Government has certain rights in the invention described herein, which was made in part with funds from the National Institutes of Health, Grant Nos. P01CA87661 (TS), R01CA85786 (TS), K01CA111343 (FG).

### FIELD OF THE INVENTION

The present invention relates generally to the latency and reactivation of cytomegalovirus (CMV) infections. In particular, the invention relates to latency promoting genomic sequences from human cytomegalovirus (HCMV) and their gene products, and methods for their use in the production of vaccines, for treatment of virus infections, and for identifying compounds useful for treating CMV infections.

### BACKGROUND OF THE INVENTION

Various publications, which may include patents, published applications, technical articles and scholarly articles, are cited throughout the specification in parentheses. Full citations for any reference not fully cited in the specification may be found at the end of the specification. Each of these cited publications is incorporated by reference herein, in its entirety.

Human cytomegalovirus (HCMV) is a ubiquitous member of the β-herpesvirus family. It has a double-stranded DNA genome of approximately 230 kilobase (kb) pairs.

A primary infection with HCMV in healthy individuals is typically clinically asymptomatic and does not cause disease. Primary infection with HCMV during pregnancy can lead to infection of the unborn child. HCMV infection in utero or in immunologically immature neonates can cause mild to severe hearing loss, physical anomalies, and cognitive deficits. Consequently, HCMV is a leading cause of infection-related birth defects.

Following the primary infection, HCMV establishes a life-long latent, i.e., substantially quiescent, infection in hematopoietic cells of the bone marrow, and possibly other cell types. Myeloid cells, in particular, monocytes, are the predominant cell-type latently-infected with HCMV (Taylor-Wiedeman et al., 1991). However, a more primitive hematopoietic progenitor cell likely serves as the primary latent reservoir for HCMV (Goodrum et al., 2002; Kondo et al., 1994; Mendelson et al., 1996; Minton et al., 1994; Reiser et al., 1986; Zhurayskaya et al., 1997).

Reactivation of HCMV from latency to productive viral infection often causes life-threatening disease in immunosuppressed individuals, such as bone marrow and solid organ transplant recipients, or individuals with AIDS. Reactivation is associated with cellular differentiation (Soderberg-Naucler et al., 1997, 2001; Taylor-Wiedeman et al., 1994), inflammation accompanying allogenic transplantation (Boeckh et al., 2003), chronic graft rejection, restinosis, and atherosclerosis (Horvath et al., 2000).

During a lytic infection, HCMV expresses over 200 genes in an ordered cascade of gene expression promoting viral replication. The first genes expressed following infection with CMV, the immediate-early genes, function as transcriptional transactivators. Following immediate-early transactivation, early genes are expressed and mediate viral DNA synthesis. Late genes are expressed following viral DNA synthesis. The late genes primarily encode structural proteins that are assembled into virus particles (Mocarski and Courcelle, 2001). Most of the genes that function in the lytic infection have been identified, and at least partially characterized.

There are ample published sequence data for HCMV. The entire genome of the human cytomegalovirus has been sequenced from several strains. For example, Murphy, Yu et al. (2003) sequenced bacterial artificial chromosomes containing the genomes of six HCMV strains. Two of the strains were laboratory strains of HCMV that were extensively passaged in cultured fibroblasts (AD169, and Towne), and four were from clinical isolates that had been passaged to only a limited extent in culture (Toledo, FIX, PH, and TR). The full genomic sequence for AD169 is available from the NCBI database (GenBank Accession No: NC_{—}001347). Even more recently, Dolan et al. (2004) sequenced a low passage strain, Merlin, and compared it to the published AD169 sequence.

The sequencing studies demonstrate that a contiguous region of approximately 15 kb is retained in all clinical isolates but lost from AD169 and other laboratory-adapted strains. The missing ˜15 kb region encodes as many as 20 genes, and the ability of strains lacking it to cause disease is attenuated (Cha et al., 1996). The majority of laboratory studies of HCMV have used the AD169 strain of CMV because it is more readily grown in culture than clinical isolates. The sequence is apparently lost during repeated serial passage of laboratory strains in cultured fibroblasts, whereas that region is retained in all clinical isolates that have been sequenced (Murphy, Yu et al., 2003, Dolan et al., 2004). These studies also demonstrated that the laboratory strains contain many additional sequence changes that could alter the function of genes outside of the 15 kb deleted region.

In addition to the genomic sequence data, there are detailed in silico studies of the structure and function of the known proteins and predicted open reading frames encoded in the virus. Novotny et al. (2001) employed a protein sequence threading approach to examine the protein sequences for folds and other structural traits for over 200 predicted ORFs in the AD169 genome, as well as additional ORFs from the Towne and Toledo strains. They were able to establish structural hypotheses for about half of the coding regions. Rigoutsos et al. (2003) used a different approach using pattern-based sequence alignment to reassess the structural character of the HCMV proteome. They confirmed and extended earlier suggestions that a significant number of proteins encoded were membrane-associated, in addition they predicted many had putative posttranslational modification sites. They also determined that many of the amino acid sequences were unique to HCMV or to herpesviruses. Their results are published in an interactive form on the IBM Bioinformatics and Pattern Discovery Group's webpage “Bio-Dictionary-based Annotations of Complete Genomes” (presently at http://cbcsrv.watson.ibm.com/virus/home.html).

HCMV disease following reactivation in transplant patients is typically treated using antiviral agents such as ganciclovir (Mocarski and Courcelle, 2001; Pass, 2001). These drugs block virus replication. Thus, currently, it is only possible to treat the symptoms of HCMV infection; it is not possible to cure an infection because the drugs that block viral replication do not target latent virus. The factors, viral and cellular, that govern the latent infection remain unknown, and to date, there have been no effective vaccines for HCMV. Genes that function to establish or maintain a latent infection are of significant interest because these genes could serve as targets for antiviral therapies, or be a basis for new vaccines or other immunomodulatory agents.

Several experimental systems have been established to study HCMV latency and latency-associated factors. Latent HCMV and several small latency-associated virus-coded transcripts have been detected in granulocyte-macrophage progenitor cells (Kondo et al., 1994; 1995; 1996). The latency-associated transcripts proved to be dispensable for establishing latency in vitro (White et al., 2000). In addition, a latency-associated transcript that is antisense to UL81-82 of HCMV was detected in monocytes from healthy, seropositive individuals (Bego et al., 2005). This transcript is intriguing since it is antisense to the mRNA encoding the essential UL82 protein and could potentially inhibit its expression (Cantrell et al., 2005; Saffert et al., 2006) although a function has yet to be demonstrated.

Antagonizing the function of genes associated with latency may inhibit the virus from establishing latency or force the virus to reactivate from latency, which in conjunction with existing antiviral treatment (i.e., ganciclovir), may facilitate viral clearance. This type of treatment would benefit CMV-seropositive individuals awaiting bone marrow, or solid organ transplantation. Alternatively, viral variants lacking the ability to produce functional products of latency promoting genes would be of utility in the development of a vaccine to CMV. Previous vaccine attempts may have failed because the laboratory strains used lacked many genes of undefined function within the 15 kb deleted region, and many other genes that were retained in the virus were probably modified in a more subtle manner during serial passage in fibroblasts. Presumably the virus was too attenuated and thus, resulted in suboptimal or inadequate immunogenicity to serve as an effective vaccine.

### SUMMARY OF THE INVENTION

In various aspects described below, the invention provides latency promoting genes from human cytomegalovirus (HCMV) and their gene products, and methods for their use in the production of vaccines, and for identifying compounds useful for treating CMV infections.

One aspect of the invention features a human cytomegalovirus comprising a wild-type genome, for example, the genome of a clinical isolate, with an alteration consisting essentially of an inoperable or missing genomic segment including one or more of UL138, UL140, UL141, and UL142. In one embodiment, the alteration is a deletion of an operable genomic segment consisting essentially or only of one or more of UL138, UL140, UL141, and UL142, or protions thereof. In certain embodiments, less than about 10 kb is missing from the wild-type sequence, and in other embodiments, less than about 5 kb is missing from the wild-type sequence.

Another aspect of the invention features a human cytomegalovirus for production of vaccines comprising an altered ability to enter or maintain a latent state, wherein the virus is deficient in a functional genomic segment including one or more of UL138, UL140, UL141, and UL142. In one presently preferred embodiment, the virus is deficient in a functional genomic segment that consists solely or essentially of one or more of UL138, UL140, UL141, and UL142, or portions thereof. In certain embodiments, the gene product is unable to perform its function in connection with entering or promoting a latent state in an infected cell. In other embodiments, the gene product for one or more of UL138, UL140, UL141, and UL142 is not produced in an infected cell.

Another aspect of the invention features a human cytomegalovirus for production of vaccines comprising an altered ability to enter or maintain a latent state, wherein the virus is deficient in a functional genomic segment including one or more of UL138, UL140, UL141, and UL142, and, in addition, other genes or coding regions are altered to make the virus a more desirable vaccine candidate, such as further attenuating the replication and spread of the virus. In certain embodiments of this aspect of the invention the virus is deficient in functional genomic segment which consists essentially or only of one or more of UL138, UL140, UL141, and UL142, or portions thereof.

Also provided herein are methods of identifying a compound useful in the treatment of human cytomegalovirus infection. Compounds that block the expression or function of a latency promoting genomic sequence that includes one or more of UL138, UL140, UL141, and UL142 could force the virus to exit latency or prevent the virus from entering latency. In certain embodiments the latentcy promoting genomic sequence consists essentially of one or more of UL138, UL140, UL141, and UL142, or protions thereof. In certain embodiments, the methods are designed to identify compounds, for example, which influence, in any one of several ways, the function or expression of the coding region from the viral genome. In other embodiments, the methods are directed to identifying compounds that alter the function or activity of the gene product, even if it is expressed in the infected cell. In certain embodiments, the identification methods comprise the use of a cell containing nucleic acids encoding the gene products of one or more of UL138, UL140, UL141, and UL142. In other embodiments, the cell is infected with a human cytomegalovirus. In presently preferred methods of identifying compounds useful for treatment of CMV infections, the infected cell is a human cell, even more preferably, the infected cell is human and of hematopoietic origin. In yet other embodiments, a viral latency-promoting protein is assayed for inhibitory effects of compounds in a cell extract. This could be an extract of any cell in which expression of the protein can be engineered, or in a partially or fully purified form.

These and other aspects of the invention will be described in more detail below and with reference to the examples, which are illustrative of several aspects of the invention, yet do not encompass the entirety of the invention, which the skilled artisan will plainly understand is capable of variation and alteration within the meaning and scope of the appended claims.

### BRIEF DESCRIPTION OF THE DRAWINGS

FIG. 1: A schematic depiction of the organization of the unique open reading frames for the FIX strain of CMV, together with mutations (thick black bars) that were introduced into this region for analysis of latency phenotypes.

FIG. 2: Low-passage quasi-clinical and clinical strains of HCMV, but not laboratory strains, establish a latent infection in CD34+ cells infected in vitro.

Panel A: Results of assay for reactivation from latency. The frequency is 1 over the number of cells required to yield one infectious center. The bars represent the average frequency of infectious centers formation in the reactivation experiment (black) or the lysate control (gray) as described in the text (four independent experiments for FIXpar, Toledo, and AD169; two independent experiments for Towne). The standard error of the means is shown for experiments with three or more replicates. The numbers above each pair of bars represents the -fold increase in infectious centers formation in the reactivation compared to the lysate control. The dashed line marks the limit of detection (LD) for the assay.

Panel B: Results of microarray assay for HCMV gene expression. HCMV gene expression in CD34+ cells infected with Toledo or AD169 strains. Arabidopsis cDNA spots used as positive controls are circled. Cellular cDNA spots used for normalizing arrays are boxed. All other spots represent HCMV genes expressed as determined using an HCMV array.

FIG. 3: ULb′ sequences promote a latent infection in vitro.

Panel A: Schematic representation of experiments wherein the kanamycin resistance gene was substituted for large regions or individual ORFs within the ULb′ region by linear recombination into the FIX strain cloned as a BAC. Black bars represent the region missing in the recombinant virus and the nucleotides missing are shown to the right of each variant.

Panel B: The latency phenotype of each substituted virus. The frequency of infectious center formation is 1 over the number of cells required to yield one infectious center. The bars represent the average frequency of infectious centers formation in the reactivation experiment (black) or the lysate control (gray). The standard error of the means is shown for experiments with three or more replicates. The numbers above each pair of bars represents the -fold increase in infectious centers formation in the reactivation compared to the lysate control.

Panel C: The latency phenotype of the kanamycin-resistance substituted virus strains. The bars represent the fraction GFP+ wells in the reactivation experiment (black) or the lysate control (gray). The standard error of the means is shown for experiments with three or more replicates.

### DETAILED DESCRIPTION OF ILLUSTRATIVE EMBODIMENTS

In accordance with various aspects of the present invention, certain molecular determinants employed by human cytomegalovirus (HCMV) to establish and/or maintain latency and reactivate from latency have been identified and elucidated. All of the experiments were conducted in well-defined, primary human hematopoietic progenitor cells infected prior to ex vivo culture. Segments of the viral genome that are required for the establishment and/or maintenance of viral latency in CD34+/CD38− hematopoietic progenitor cells have been identified. Several specific segments have been found to be required to establish and/or maintain a latent infection.

The discoveries leading to the various aspects of the invention were made in part through the use of a unique system in which to analyze HCMV infection and latency in primary hematopoietic cells. In this system, populations of hematopoietic cells enriched for expression of the CD34 cell surface progenitor marker are infected directly following their isolation from human bone marrow. Periods of cell culture before infecting CD34+ cells are avoided, thus minimizing ex vivo stimulation that might alter the natural differentiation state of this dynamic cell population. Following infection, populations of infected cells are isolated by fluorescent-activated cell sorting (FACS), based on expression of a gene encoding a green fluorescent marker protein (GFP) that had been engineered into the viral chromosome (Goodrum et al., 2002; Goodrum et al., 2004). The sorted cells are then placed into long-term culture over a stromal cell monolayer that helps to maintain progenitor cell populations (Hogge et al., 1996; Lewis et al., 2001; Miller and Eaves, 2002; Nolta et al., 2002).

Using the foregoing system, the inventors have shown that progenitor cells expressing the CD34+/CD38− cell surface phenotype express a limited set of HCMV genes following infection. This set includes genes normally expressed during the immediate-early, early, and late phases of lytic infection. However, widespread expression of HCMV genes is transient. Viral genomes are maintained for extended periods of culture, but after an initial phase of viral gene expression, viral mRNAs are not detected. At 10 to 14 days post infection, infected hematopoietic cells may be transferred to co-culture with human fibroblasts that are permissive for HCMV infection. This reactivates viral replication in about one in 15,000 to 20,000 infected cells (Goodrum et al., 2004). Thus, the culture system exhibits the hallmarks of viral latency: the virus infects cells, viral DNA is maintained with little or no viral gene expression, and the virus can be reactivated to replicate and produce progeny in response to specific stimuli.

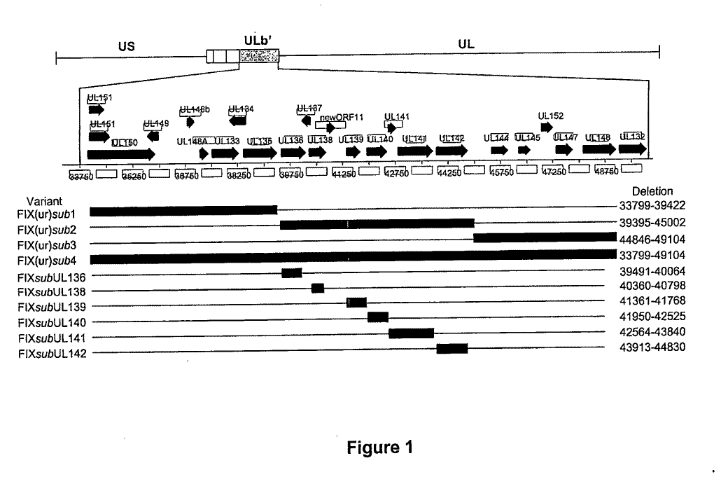

In recent studies, the relative ability of AD169 (a highly passaged laboratory strain), Toledo (a quasi-clinical, low-passage, strain), and FIX (a clinical strain), to replicate in hematopoietic progenitor cells was compared. From these studies, and studies using virus strains engineered to lack specific sequences within the ˜15 kb unique region (i.e., the region lost from AD169, FIG. 1), specific genomic segments that function in establishing or maintaining a latent infection were identified. These genomic segments are believed to function during the establishment of latency, because that is the time at which the transient accumulation of viral mRNAs was detected. However, by way of explanation and not to limit the invention, a maintenance role for these genomic segments can not be completely ruled out, because they could be continuously expressed at a level below the current detection limit for their mRNAs. The results are summarized in greater detail in the Example.

Various embodiments of this invention encompass important biomedical applications that follow from the identification of genomic segments that function in viral latency. One application is in the area of vaccines. Deletion of one or more of the latency-promoting genomic segments in HCMV, or interference with the function of the gene products encoded by these segments, produces a virus unable or with reduced capacity to enter latency, but which still retains the genes needed for infecting and replicating in the cell and thus, inducing protective immunity. Such a strain is much safer as an immunizing agent than a virus retaining the ability to enter a latent stage. Also, the inability to enter latency might make the virus more immunogenic, because it will have lost one of its principle mechanisms for escaping detection by the immune system. Further, such a virus will lack only the genes required for latency and not the many other functions that have been altered in over-attenuated laboratory strains, which may be important for infecting the appropriate cells and inducing immunity. If necessary, additional selected mutations can be added to virus strains lacking one or more latency-promoting genomic segments that will serve to further attenuate the virus for vaccine purposes without over-attenuating the virus. Prior to the development of the virus strains provided herein, previous vaccine candidates were over-attenuated laboratory strains, which functioned poorly as vaccines. The present invention solves this problem by providing a safe and effective vaccine strain.

As another example, the products of these genomic regions can serve as targets for drugs that antagonize their function. The anticipated result is that the virus would reactivate a lytic program of viral replication when a patient is treated with such drugs. The virus could not replicate and spread if the activation was induced in the presence of a second drug, such as ganciclovir, that prevents viral DNA replication. Drug-induced activation of latent HCMV in the presence of ganciclovir could clear latent HCMV from an individual awaiting organ or bone marrow transplantation. This would prevent subsequent reactivation when the patient is immunosuppressed and thus, highly susceptible to HCMV disease. These and other embodiments are described in detail below.

HCMV Variants and Vaccines:

One aspect of the invention features human cytomegalovirus variants possessing a wild-type genome with an alteration comprising an inoperable or missing genomic region or segment for one or more latency-promoting genomic segments described herein, in particular, a genomic segment comprising one or more of UL138, UL140, UL141, and UL142, or protons thereof. In presently preferred embodiments, the human cytomegalovirus comprises a deletion of an operable genomic region for one or more of UL138, UL140, UL141, and UL142, or portions thereof.

As used herein, “genomic segment” refers to any portion of a genome comprising one or more genes, ORFs, or the like, or combinations or portions thereof. Sometimes the term “genomic region” is used essentially synonymously therewith.

As used herein a “wild-type” HCMV comprises a clinical strain or isolate obtained from an infected subject or diseased individual, or a strain that is capable of lytic replication and assembly of virus particles, as well as fully capable of entering and maintaining a latent stage. Wild-type HCMVs can be maintained in culture, and need not be primary isolates per se, however, preferably they have not been highly passaged as that term is used in the art, and even more preferably, they have retained their full genome. In more preferred embodiments, the wild-type HCMV expresses a full proteome under appropriate conditions. Such HCMVs have been studied in detail and are thus known in the art—examples include, but are not limited to Merlin (Dolan et al. (2004)), Toledo, FIX, PH, and TR (Murphy, Yu et al. (2003)). Preferably the genome of a wild-type HCMV (a “wild type genome” as used herein) comprises a full array of the genes found in clinical isolates of HCMV. More preferably it is at least about 225-240 kb in size. Still more preferably a wild-type genome is about 230-235 kb in size. Such genomes are known in the art, for example, but not limited to, those for FIX, Merlin (GenBank Accession No: NC_{—}006273), PH, and TR.

As used herein, “alteration” comprises any change including alterations, changes, and mutations, as well as addition or deletion of a sequence or sequences. For the production of useful vaccine viruses, it is preferable in certain embodiments to alter a genomic segment comprising one or more of UL138, UL140, UL141, and UL142 so as to inactivate their coding regions and/or gene products, and to alter certain other genes so as to reduce the pathogenicity of the virus. Although the one or more genomic regions coding for UL138, UL140, UL141, and UL142, and other sequences may be missing entirely, it is not intended that substantially larger portions of the genome, relative to the specified genes, should be missing from the HCMV as provided herein. Such viruses, e.g. AD169, which are missing large portions of genome (e.g 15 kb) are previously known in the art and have proven not to be useful, for example, for the production of vaccines. Preferably, the viruses of the invention in this aspect are generally useful as vaccines or in the production of same, and are distinguishable from the art-known strains, for example laboratory strains with large deletions of a great many genes with unrelated functions. Thus in certain embodiments of each aspect of the invention described herein, the latentcy-promoting genomic segment, or altered or missing genomic region is said to consist essentially of, or in some cases solely of, UL138,UL140, UL141, and UL142—to help distinguish the contructs and virus of the instant invention from the art-known virus with large delections not necessarily related to latency, pathogenicity, vaccine construction and the like.

As used herein “UL138” refers to a unique long (UL) ORF from HCMV (also known as human herpesvirus 5 or HHV5) of about 500-520 nucleotides and preferably about 510 nucleotides in length. The open reading frame potentially encodes a protein of about 165-175 amino acid residues in length, and preferably about 169 amino acids in length. It is possible that the coding region functions at the level of DNA or RNA, and it also is possible that a portion or all of the coding region is joined to other coding regions as a consequence of splicing. The presently preferred encoded sequences are about 169 amino acids in length. Nucleotide sequences for UL138 are known in the art, and provided, in whole or in part, in public databases such as those at the National Center for Biotechnology Information (NCBI). For example, UL138 sequences are provided at GenBank Accession Nos. AY941165, AY941164, AY194163, AY601873, AY446894, AY446871, AY446870, AY446869, AY446868, AY446867, AY446866, AY446865, AY446864, AY255773, AY255772, and AY218872 through AY218860.

As described above for UL138, UL140, UL141, and UL142 refer to additional unique long (UL) ORFs from HCMV (also known as human herpesvirus 5 or HHV5). They are located in the vicinity of UL138 (FIG. 1), and the ORFs are defined in Murphy, Yu et al. (2003) and Dolan et al., (2004), each of which is incorporated by reference in its entirety herein. For each of these ORFs, it is possible that a portion or all of the coding region is joined to other coding regions as a consequence of splicing.

Thus, as used herein “UL140” refers to a unique long (UL) ORF from HCMV of about 350-600 nucleotides in length, and preferably about 585-595 nucleotides in length. The open reading frame encodes a protein of about 185-195 amino acid residues in length. Presently preferred encoded sequences are about 191 amino acids in length. Nucleotide sequences for UL140 are known in the art, and provided, in whole or in part, in public databases such as those at NCBI. For example, UL140 sequences are provided at GenBank Accession Nos. NC_{—}006273, DQ229942 through DQ229948, DQ180385, DQ180373, DQ180357, AY446894, AY446871 through AY446864, AF480884, AY255774 through AY255777, AY218849 through AY218859, and U33331 (reported as 114 amino acids encoded by 344 bp, from Toledo strain).

As used herein “UL141” refers to a unique long (UL) ORF from HCMV of about 1250-1300 nucleotides in length, and preferably about 1265-1272 nucleotides in length. The open reading frame encodes a protein of about 330-340 amino acid residues in length. Presently preferred encoded sequences are about 338 amino acids in length. Nucleotide sequences for UL141 are known in the art, and provided, in whole or in part, in public databases such as those at NCBI. For example, UL141 sequences are provided at GenBank Accession Nos. AY941105, AY941104, AY600459 through AY600468, AY446894, AY446871 through AY446864, AY496547 through AY496555. In addition, examples of sequences of UL141 which are nonfunctional due to mutation are published at GenBank Accession Nos. AY366466 through AY366472.

As used herein “UL142” refers to a unique long (UL) ORF from HCMV of about 850-950 nucleotides in length, and preferably about 910-930 nucleotides in length. The open reading frame encodes a protein of about 295-315 amino acid residues in length. Presently preferred encoded sequences are about 306 amino acids in length. Nucleotide sequences for UL142 are known in the art, and provided, in whole or in part, in public databases such as those at NCBI. For example, UL142 sequences are provided at GenBank Accession Nos. AY217037 through AY217045, AY446871 through AY446864, AY446894, AY941173, AY941174, AY941175, DQ229942 through DQ229948, DQ180370, DQ180384, NC_{—}006273, DQ453517, DQ453518, DQ457001, and U33331.

Certain embodiments of the invention comprise human cytomegaloviruses for production of vaccines, wherein the viruses comprise an altered ability to enter or maintain a latent state, wherein the viruses are deficient in functional gene product including one or more of UL138, UL140, UL141, and UL142. In certain preferred embodiments, the gene product for one or more of UL138, UL140, UL141, and UL142 is not functional, or not sufficiently functional to facilitate the virus entering a latent state, or for promoting or maintaining the virus in a latent state in an infected cell. Also preferred are embodiments wherein the human cytomegalovirus described does not produce the gene product including, or in some cases limited essentially to or soley to, one or more of UL138, UL140, UL141, and UL142 in an infected cell. It is to be understood that in certain embodiments, a latentcy-promoting or other gene product as contemplated herein may comprise coding portions from one or more ORFs or genes including UL138, UL140, UL141, and UL142, for example, where various gene splicing events occur.

The phenotype exhibited by the above-described viruses comprises, in part, an altered ability to enter or maintain a latent state. Preferably, the ability of the virus to enter or to maintain a latent state is lacking altogether. In certain other preferred embodiments, the phenotype of the virus is further characterized in having a reduced pathogenicity, for example, a decreased ability to replicate in the lytic phase, or to spread from cell to cell. This phenotypic characteristic is in addition to the altered ability to enter or maintain a latent state. The viruses preferably comprise a deficiency of functional gene product of one or more latency promoting genes, in particular including one or more of UL138, UL140, UL141, or UL142. In various embodiments the deficiency consists essentially, or even solely, of one or more of UL138, UL140, UL141, or UL142, or portions thereof. The deficiency of functional gene product can result from the genomic sequence being completely missing or deleted, from a disruption of the coding region by mutation (for example one or more insertions, deletions, substitutions, any change that results in a missense or nonsense mutation), or from altered or nonfunctional regulatory sequences or sequences required for proper transcription or translation of a gene product from the virus.

The viruses preferably have a genotype which comprises a wild-type genome with an alteration consisting essentially of an alteration of one or more latency-promoting genes having a sequence which is at least 80, 85, or 90% identical to a UL138, UL140, UL141, or UL142 genomic sequence. Even more preferred are those genes with sequences which are more than 90% identical to a UL138, UL140, UL141, or UL142 gene sequence, especially those that are 92, 93, or 94% identical. Still more preferred are those sequence which are 95% or more identical to a UL138, UL140, UL141, or UL142 gene sequence. In other embodiments the genotype of the vaccine strain further comprises one or more alterations in additional genes, wherein the alterations attenuate the lytic replication or spread of the virus. The genotype of the viruses provided herein specifically confers the advantage of greater efficacy and safety. The strains are unable to enter or maintain a latent state, and thus offer improved opportunities to be recognized and generate response from a subject's immune system, thereby conferring enhanced protection against future HCMV infection. Also, due their inability to establish or maintain a latent infection, they can safely be used in patients relative to HCMV strains which retain that ability.

Assays:

Also provided herein are methods of identifying compounds useful in the treatment of human cytomegalovirus infection by virtue of their ability to modulate production or function of the gene products of one or more HCMV latency-promoting genes, including UL138, UL140, UL141, and UL142. For example, these assays may be adapted to identify compounds that affect expression of one or more of the latency-promoting genes, e.g., by affecting transcription or translation of the gene. Other assays may be adapted to identify compounds that modulate the function or activity of the gene product produced by expression of a latency-promoting gene.

Thus, in one embodiment, an assay is provided for identifying compounds that directly or indirectly affect expression of specific CMV genes involved in establishing or maintaining a viral latent state. Such methods utilize the steps of (1) providing an expression system comprising one or more HCMV latency-promoting genes, including at least a portion of the region coding UL138, UL140, UL141, and UL142; (2) in the presence or absence of a test compound, subjecting the expression system to conditions that, in the absence of test compound (control), allow for the expression of the nucleic acid sequences; (3) measuring a parameter indicative of expression of the nucleic acid sequences in the presence of the test compound (and in the control); and (4) determining the effect of the test compound on expression of the latency-promoting gene by comparing the measurement in the presence of the test compound to that in the control. In presently preferred embodiments, the expression system comprises a human cell. Specific embodiments are directed to methods wherein the cells are hematopoietic cells. Preferably, the nucleic acid sequences used in the foregoing methods comprise one or more regulatory sequences associated with UL138, UL140, UL141, and UL142 expression in a human cytomegalovirus. Compounds capable of altering the regulation of such sequences may be particularly useful in treating aspects of the viral infection associated with CMV.

In other embodiments, methods are provided for identifying a compound useful in the treatment of human cytomegalovirus infection in a subject, wherein the compounds affect the function of a gene product required for entering or maintaining a viral latent state. Typically, the methods comprise (1) providing a gene product encoded at least in part by one or more of UL138, UL140, UL141, or UL142, or portions thereof; (2) providing a measurement of an amount of function for at least one of the gene products; (3) measuring the amount of function at least one of the gene products in the presence of a test compound, (and in a control in the absence of the test compound), under conditions which, in the absence of the test compound, allow for the function of the gene products being measured; and (4) determining if the compound alters the amount of function measured for the at least one gene product by comparing the amount of function in the presence of the test compound to that in the control, thereby identifying a compound useful in treating a human cytomegalovirus infection. In certain preferred embodiments, the gene product is provided in an expression system. In some embodiments the method is conducted in an infected cell, or a cell fraction or an extract from such a cell. For example, the use of mammalian, yeast, or bacterial cells is contemplated herein. In other embodiments, the gene products are provided in vitro in either a crude, partially purified, or purified form.

Another assay to identify compounds useful for the treatment of a human cytomegalovirus infection comprises the steps of: (1) providing cells infected with a human cytomegalovirus including UL138, UL140, UL141, and UL142; (2) incubating the infected cells in the presence of a test compound, and as a control, incubating another population of the cells in the absence of the test compound, under conditions which, in the absence of the test compound, allow for entry into or maintenance of a viral latent phase; (3) measuring, directly or indirectly, the transcription of at least one of UL138, UL140, UL141, or UL142, or translation of the transcript of at least one of UL138, UL140, UL141, or UL142; and (4) correlating the ability of the virus in the infected cell incubated in the presence of the test compound relative to that of the control to enter into or maintain the viral latent phase with the relative amount of transcription or translation at least one of UL138, UL140, UL141, and UL142. In presently preferred methods the infected cell is a human cell, even more preferably, the infected cell is hematopoietic.

The following example is provided to describe the invention in greater detail. It is intended to illustrate, not to limit, the invention.

Identification of HCMV Genetic Elements that Promote Latency.

An experimental latency system using primary CD34+/CD38− hematopoietic progenitor cells that are infected (Goodrum et al., 2004) with HCMV was developed. In these cells, HCMV transiently expressed a unique subset of viral genes in the absence of substantial virus replication. Virus can be reactivated from these cells.

HCMV strains that have been adapted for growth in cultured fibroblasts have acquired multiple genome rearrangements that distinguish them from clinical and low-passage virus strains. In particular, laboratory strains have lost a 13-15 kilobase (kb) DNA segment that is retained in all clinical strains (Cha et al., 1996; Dolan et al., 2004; Murphy, Yu et al., 2003). This region, representing the majority of the ULb′ region, encodes as many as 20 open reading frames (ORFs) including UL133-UL151 (FIG. 1). These sequences are dispensable for virus replication in fibroblasts. Indeed, laboratory strains replicate more efficiently and to higher titers thatn clinical isolates in these cells.

Using the experimental model for latency described above, the relative abilities of the FIX and Toledo clinical strains to establish latent infections in CD34+ cells were compared to those of the Towne and AD169 laboratory strains. The FIX and Toledo strains both retain the ULb′ region, although the region in Toledo is inverted relative to other clinical strains (Cha et al., 1996). AD169 and Towne strains lack 15.2 and 13.1 kb of the the ULb′ region, respectively (Prichard et al., 2001).

Methods:

Following isolation from bone marrow or cord blood, CD34+ cells were infected with one of the clinical HCMV strains used (FIX or Toledo) or with one of the laboratory strains (Towne or AD169) without prior ex vivo culture and purified by fluorescent-activated cell sorting (FACS). Each of the strains used in our studies was engineered to express the green fluorescent protein (GFP) (Goodrum et al., 2002). Pure populations of infected (GFP+) CD34+ cells were cultured on murine stromal cells, which maintain progenitor cell phenotype and function (Miller and Eaves, 2002). Following 10-14 days in culture, infected hematopoietic cells were transferred by limiting dilution into co-culture with permissive fibroblasts to reactivate viral replication. As a control, an equivalent number of infected CD34+ cells were mechanically lysed prior to seeding onto fibroblasts to distinguish infectious centers formed as a result of reactivation from those produced during the culture period prior to reactivation.

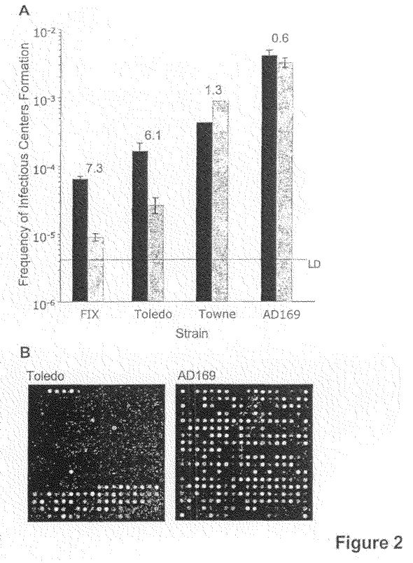

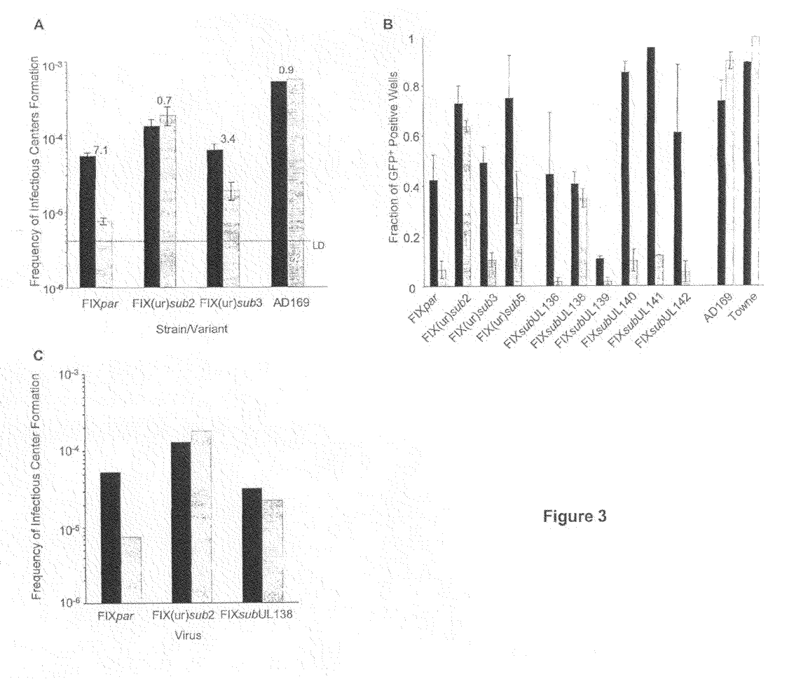

Infectious center formation by each of the HCMV strains was quantitatively compared. Limiting dilution analysis was used to determine the frequency of infectious centers formation in CD34+ cells infected with the clinical strains (FIX and Toledo) or the laboratory strains (Towne and AD169) at 10-14 days post infection. Infected cells, or an equivalent cell lysate, were serially diluted into co-culture with permissive fibroblasts in 96 wells dishes. Twenty-four days later, the fraction of GFP+ wells for each dilution was scored. Results are shown in FIG. 2A and expressed as average frequency of infectious centers formation in the reactivation experiment (black) or the lysate control (gray) in four independent experiments for FIXpar, Toledo, and AD169, and in two independent experiments for Towne.

Viral gene expression was determined in CD34+ cells infected with Toledo or AD169 strains. Cells infected at a multiplicity of 5 PFU/cell were purified 20 hours after infection by FACS and seeded into long-term bone marrow cultures. Linearly-amplified RNA was analyzed using the HCMV array at 30 days post infection. All RNAs were hybridized in triplicate. The results are shown in FIG. 2B.

To determine if sequences within the ULb′ region unique to clinical strains contributed to viral latency, a cassette encoding kanamycin was substituted for large segments within the ULb′ region (FIX(ur)sub1-4) of the FIX strain (FIG. 1). FIX(ur)sub2 and 3, which grew normally in cultured fibroblasts (data not shown), were analyzed for their ability to establish a latent infection in CD34+/CD38− cells relative to the parental FIX (FIXpar) and AD169 strains (FIG. 3A).

To more specifically determine if specific ORFs were required to establish or maintain a latent infection in vitro the cassette containing the gene encoding kanamycin resistance was substituted for individual open reading frames (FIG. 1). Each of the resultant viruses was analyzed for its ability to establish a latent infection in CD34+/CD38− cells using a modified reactivation assay where 10,000 infected CD34+/CD38− cells, or an equivalent cell lysate, were seeded into each of 24 wells of a 96-well dish containing permissive fibroblasts. At 24 days post infection, the fraction of wells with GFP+ fibroblasts was scored (FIG. 3B).

Results:

The clinical strains, FIX and Toledo, produced fewer infectious centers compared to the laboratory strains, AD169 and Towne (FIG. 2A). Formation of an infectious center required 15,291 cells infected with FIX, 6,012 cells infected with Toledo, 1,818 cells infected with Towne, or 241 cells infected with AD169. These results indicate that increased adaption to growth in cultured fibroblasts correlates to more efficient replication in hematopoietic progenitors. Interestingly, the clinical strains produced 6-7 fold more infectious centers in the reactivation assay compared to the lysate control whereas the laboratory strains generated similar amounts of infectious virus with or without a reactivation stimulus. Prior to reactivation, one cell in 111,421 or 36,765 cells infected with FIX or Toledo, respectively, produced and infectious center prior to reactivation. By contrast, Towne and AD69 produced infectious centers in one in 1,111 or 308 cells, respectively, resulting in a reactivation/lysate ratio close to one. These results demonstrate that clinical strains, but not laboratory strains, can establish and maintain an infection in CD34+ cells consistent with latency.

The results for the viral gene expression in CD34+ cells infected with Toledo or AD169 observed using our HCMV array are shown in FIG. 2B.

It has been found previously that CD34+/CD38− cells infected with the Toledo strain transiently express a unique subset of viral RNAs following infection (Goodrum et al., 2004). The RNAs detected were encoded by early and late kinetic classes of genes. Many of the RNAs have no known function or are not thought to encode a protein. Expression of these RNAs was not detected by 8-10 days post infection, a time when viral genomes were most abundant.

As can be seen in FIG. 2B, at 20 days postinfection, no viral gene expression was detected in CD34+ cells infected with Toledo. AD169, however, expressed its genes as would be expected for a lytic infection. These results are consistent with those for the latency/reactivation assay shown in FIG. 2A.

Sequences within the ULb′ region unique to clinical strains (FIG. 1) contributed to viral latency. Of the strains having the cassette encoding kanamycin resistance substituted for segments within the ULb′ region, FIX(ur)sub1 and 4 were defective for growth (data not shown), Strikingly, FIX(ur)sub2 produced high levels of infectious centers in both the reactivation assay and lysate similar to AD169 (FIG. 3A). One in 7,498 cells and 1 in 5,402 cells infected with FIX(ur)sub2 produced an infectious center in the reactivation and lysate assays, respectively. By contrast, FIX(ur)sub3 produced 3-fold greater infectious centers in the reactivation as in the lysate, a phenotype more closely resembling the FIXpar strain (FIG. 3A). One in 15,654 cells infected with FIX(ur)sub3 were required to form an infectious center upon reactivation compared to 52,809 cells in the lysate control. From these data, we conclude that sequences encoded within UL136-UL142 are required for the establishment and/or maintenance of a latent infection in vitro.

HCMV genomic sequences encoding one or more specific ORFs were required to establish or maintain a latent infection in vitro, By substituting the gene encoding kanamycin resistance for individual open reading frames (FIG. 1) it was determined that UL 138 was required to establish or maintain a latent infection in vitro. With the exception of FIXsubUL136, which exhibited a modest growth defect, each of the substitution viruses grew with wild-type kinetics in cultured fibroblasts (data not shown). FIXpar and FIX(ur)sub3 exhibited a latency phenotype where a greater fraction of wells had infectious centers in the reactivation compared to the lysate control (FIG. 3B). Only the FIXsubUL138 virus produced nearly equivalent numbers of infectious centers in the lysate as in the reactivation assay. This was similar to the results seen with the virus strain FIX(ur)sub2, as well as the laboratory strains. All of the other single-ORF substitutions resulted in a wild-type latency phenotype (FIG. 3B). When further analyzed in a limiting dilution format, FIXsubUL138 produced infectious centers in 1 in 30,063 cells in the reactivation and 1 in 43,304 cells in the lysate control (FIG. 3C). These results confirmed that sequences encoding UL138 promote a latent infection in vitro. It is possible that additional ORFs included in the region deleted in FIX(ur)sub2 also sontribute in a subtle manner to latency, even though the present assay for individual ORF deletions is not able to detect their effect. Importantly, the deficiency displayed by FIXsubUL138 in the reactivation assay is believed to be the first functional demonstration of an HCMV sequence required to promote a latent infection.

From viral array and PCR experiments, UL138 is expressed in productively infected fibroblasts (data not shown), and in CD34+/CD38− cells infected experimentally (Goodrum et al., 2002; Goodrum et al., 2004). There is more than one mechanism by which UL138 could promote the establishment and maintenance of a latent infection. The UL138 transcript could be a functional RNA, similar to the latency-associated transcript of herpes simplex virus, or it may encode a protein (Gupta et al., 2006). It is also conceivable that UL38 exerts its function through the DNA itself, rather than through an expressed product. Based at least in part on the evidence described herein, it is believed to be more likely that UL138 encodes a protein or a portion of a protein. UL138 has a 5′ ATG, a Kozak motif, is predicted to encode a protein by the biodictionary-based gene-finder algorithm, and is conserved in chimpanzee CMV (Murphy, Rigoutsos et al., 2003).

The U94/REP protein of human herpesvirus-6 (HHV-6), a member of the β-herpesvirus family distantly related to HCMV, restricts HHV-6 replication (Rotola et al., 1998) and that of HCMV (Caselli et a, 2006). The ability of U94/REP to restrict replication is likely important to HHV-6 latency. UL138 may encode a similar function to promote latency.

In growth curve analyses (data not shown), viruses lacking UL138 did not show a dramatic growth advantage in cultured fibroblasts relative to the parental strain, however, such an activity may be dependent on the cell type and may not score in fibroblasts.

These results described in the foregoing example has identified UL138 as a gene having a prominent role in establishing and/or maintaining latency. Based on the data, other genes within the ULb′ region are also expected be involved. For example, the mutant virus lacking only UL138 does not produce infectious centers in hematopoietic progenitor cells with the same efficiency as the FIX(ur)sub2 virus. Further, infection with both the FIX(ur)sub2 and the FIX(ur)sub3 viruses resulted in increased infectious center formation in the lysate control relative to FIXpar. Such results indicate that other sequences have a suppressive effect on viral replication in hematopoietic cells. While the latency phenotype of the FIX(ur)sub3 virus resembles that of FIXpar, the difference between infectious centers resulting from reactivation and those in the lysate is reduced to 3-fold (P value from paired Student's t-test is 0.04).

One explanation for the increased leakiness in infectious center formation prior to reactivation (lysate) in cells infected with FIX(ur)sub3 is that one or more sequences within UL144-UL148 may contribute to efficient maintenance of the latent infection. While all single-ORF substitution viruses other than FIXsubUL138 exhibited a wild-type phenotype, several substitutions resulted in viruses that reactivated to produce infectious centers in a greater fraction of wells compared to the FIXpar. Substitutions into UL140, UL141, and UL142 each showed this characteristic (FIG. 3B).

### REFERENCES

Bego, M:, J. Maciejewski, S. Khaiboullina, G. Pan, S. St Jeor, J Virol 79, 11022 (September, 2005).
Boeckh, M., et al., Biol Blood Marrow Transplant 9, 543 (September, 2003).
Cantrell, S. R., W. A. Bresnahan, J Virol 79, 7792 (June, 2005).
Caselli, E. et al., Virology 346, 402 (Mar. 15, 2006).
Cha, T. A., Tom, E., Kemble, G. W., Duke, G. M., Mocarski, E. S., and Spaete, R. R. (1996). J Virol 70, 78-83.
Dolan, A., Cunningham, C., Hector, R. D., Hassan-Walker, A. F., Lee, L., Addison, C., Dargan, D. J., McGeoch, D. J., Gatherer, D., Emery, V. C., Griffiths, P. D., Sinzger, C., McSharry, B. P., Wilkinson, G. W., and Davison, A. J. (2004) J Gen Virol. 85 (Pt 5):1301-1312.
Goodrum, F., Jordan, C. T., Terhune, S. S., High, K. P., and Shenk, T. (2004). Blood 104, 687-695.
Goodrum, F. D., Jordan, C. T., High, K., and Shenk, T. (2002). Proc Natl Acad Sci USA 99, 16255-16260.
Gupta, A., J. J. Gartner, P. Sethupathy, A. G. Hatzigeorgiou, N. W. Fraser, Nature 442, 82 (Jul. 6, 2006).
Hogge, D. E., Lansdorp, P. M., Reid, D., Gerhard, B., and Eaves, C. J. (1996). Blood 88, 3765-3773.
Horvath, J. et al., J Clin Virol 16, 17 (February, 2000). Kondo, K., H. Kaneshima, E. S. Mocarski, Proc Natl Acad Sci USA 91, 11879 (1994).
Kondo, K, E. S. Mocarski, Scand J Infect Dis Suppl 99, 63 (1995).
Kondo, K., J. Xu, E. S. Mocarski, Proc Natl Acad Sci USA 93, 11137 (1996).
Lewis, I. D., Almeida-Porada, G., Du, J., Lemischka, I. R., Moore, K. A., Zanjani, E. D., and Verfaillie, C. M. (2001). Blood 97, 3441-3449.
Mendelson, M. S. Monard, P. Sissons, J. Sinclair, J Gen Virol 77, 3099 (1996).
Miller, C. L., and Eaves, C. J. (2002). C. A. Klug, and C. T. Jordan, eds. (Totowa: Humana Press), pp. 123-141.
Minton, E. J., C. Tysoe, J. H. Sinclair, J. G. Sissons, J Virol 68, 4017 (1994).
Mocarski, E. S., and Courcelle, C. T. (2001). In Fields Virology, D. M. Knipe, and P. M. Howley, eds. (Philadelphia: Lippincott, Williams & Wilkins), pp. 2629-2673.
Murphy, E., I. Rigoutsos, T. Shibuya, T. E. Shenk, Proc Natl Acad Sci USA 100, 13585 (Nov. 11, 2003).
Murphy, E., Yu, D., Grimwood, J., Schmutz, J., Dickson, M., Jarvis, M. A., Hahn, G., Nelson, J. A., Myers, R. M., and Shenk, T. E. (2003). Proc Natl Acad Sci USA 100, 14976-14981.
Nolta, J. A., Thiemann, F. T., Arakawa-Hoyt, J., Dao, M. A., Barsky, L. W., Moore, K. A., Lemischka, I. R., and Crooks, G. M. (2002). Leukemia 16, 352-361.
Novotny, J., Rigoutsos I., Coleman D., and Shenk T. (2001). J Mol Biol 310, 1151-1166.
Pass, R. F. (2001). In Fields Virology, D. M. Knipe, and P. M. Howley, eds. (Philadelphia: Lippincott, Williams & Wilkins), pp. 2675-2705.
Prichard, M. N., M. E. Penfold, G. M. Duke, R. R. Spaete, G. W. Kemble, Rev Med Virol 11, 191 (May-June, 2001).
Reiser, H. et al., J Gen Virol 67 (Pt 12), 2595 (December, 1986).
Rigoutsos I, Novotny J, Huynh T, Chin-Bow S T, Parida L, Platt D, Coleman D, Shenk T. (2003). J Virol. 77, 4326-4344.
Rotola, A. et al., Proc Natl Acad Sci USA 95, 13911 (Nov. 10, 1998).
Saffert, R, T., R. F. Kalejta, J Virol 80, 3863 (April, 2006).
Soderberg-Naucler, C., K. N. Fish, J. A. Nelson, Cell 91, 119 (1997).
Soderberg-Naucler, C. et al., J Virol 75, 7543 (2001).
Taylor-Wiedeman, J., J. G. Sissons, L. K. Borysiewicz, J. H. Sinclair, J Gen Virol 72, 2059 (1991).
Taylor-Wiedeman, J., P. Sissons, J. Sinclair, J Virol 68, 1597 (1994).
White, K. L., B. Slobedman, E. S. Mocarski, J Virol 74, 9333 (2000).
Zhurayskaya, T. et al., Blood 90, 2482 (1997).

What is claimed:

1. A human cytomegalovirus comprising a wild-type genome with an alteration consisting essentially of an inoperable or missing genomic segment including one or more of UL138, UL140, UL141, or UL142.

2. The human cytomegalovirus of claim 1 wherein the alteration is a deletion of an operable genomic segment including one or more of UL138, UL140, UL141, or UL142.

3. The human cytomegalovirus of claim 1 or 2 wherein the genomic segment consists essentially of one or more of UL138, UL140, UL141, or UL142, or portions thereof.

4. The human cytomegalovirus of claims 1-3 wherein less than about 10 kb is missing from the wild-type sequence.

5. The human cytomegalovirus of claims 1-4 wherein less than about 5 kb is missing from the wild-type sequence.

6. A human cytomegalovirus for production of vaccines comprising an altered ability to enter or maintain a latent state, wherein the virus is deficient in functional gene product including one or more of UL138, UL140, UL141, or UL142, or portions thereof.

7. The human cytomegalovirus of claim 6 wherein the functional gene product consists essentially of one or more of UL138, UL140, UL141, or UL142, or portions thereof.

8. The human cytomegalovirus of claim 6 or 7 wherein the gene product is not functional for entering or promoting a latent state in an infected cell.

9. The human cytomegalovirus of claims 6-8 wherein the gene product is not produced in an infected cell.

10. A method of identifying a compound useful in the treatment of human cytomegalovirus infection comprising:
providing one or more human cytomegalovirus nucleic acid sequences consisting essentially of one or more of UL138, UL140, UL141, or UL142 in an expression system for expressing the one or more nucleic acid sequences;
exposing the expression system in the presence of a test compound, and in a control in the absence of the test compound, to conditions which, in the absence of test compound, allow for the expression of at least one of the one or more nucleic acid sequences;
measuring a parameter indicative of expression of the at least one of the one or more nucleic acid sequences in the presence of the test compound and in the control;
determining the effect of the test compound by comparing the measurement in the presence of the test compound to that in the control, thereby identifying compounds useful for the treatment of human cytomegalovirus infection.

11. The method of claim 10 wherein the expression system comprises a human cell.

12. The method of claim 10 or 11 wherein the cells are hematopoietic cells.

13. The method of claims 10-12 wherein the nucleic acid sequences comprise one or more regulatory sequences associated with expression of one or more of UL138, UL140, UL141, or UL142 in a human cytomegalovirus.

14. A method of identifying a compound useful in the treatment of human cytomegalovirus infection in a subject comprising
providing a gene product encoded at least in part by one or more of UL138, UL140 UL141, or UL142;
providing a measurement of an amount of function for at least one of the one or more gene products;
measuring the amount of function of the at least one gene product in the presence of a test compound, and in a control in the absence of the test compound, under conditions which, at least in the absence of the test compound, allow for the function of the one or more gene products;
determining if the compound alters the amount of function measured for the at least one gene product by comparing the amount of function in the presence of the test compound to that in the control,
thereby identifying a compound useful in treating a human cytomegalovirus infection.

15. The method of claim 14 wherein the step of providing the gene product comprises expressing a nucleic acid encoding the gene product.

16. The method of claim 15 wherein the expressing is in a cell.

17. The method of claims 16 wherein the cell is human.

18. The method of claims 16-17 wherein the cell is hematopoietic in origin or derived from a hematopoietic cell or tumor.

19. A method for the identification of a compound useful for the treatment of human cytomegalovirus comprising the steps of
providing a cell infected with a human cytomegalovirus comprising at least one of UL138, UL140, UL141, or UL142;
incubating an infected cell in the presence of a test compound, and as a control, incubating an infected cell in the absence of the test compound, under conditions which, in the absence of the test compound allow for entry into or maintenance of a viral latent phase;
measuring, directly or indirectly, the transcription of at least one of UL138, UL140, UL141, or UL142, or the translation of the transcript of at least one of UL138, UL140, UL141, or UL142; and
correlating the ability of virus in the infected cell incubated in the presence of the test compound relative to that of the control to enter into or maintain the viral latent phase with the relative amount of transcription or translation of at least one of UL138, UL140, UL141, or UL142,
thereby identifying a compound useful for the treatment of human cytomegalovirus.

20. The method of claim 19 wherein the infected cell is a human cell.

21. The method of claims 19-20 wherein the infected cell is hematopoietic.

22. A human cytomegalovirus for production of a vaccine, the virus phenotype comprising an altered ability to enter or maintain a latent state, the virus comprising a deficiency of functional gene product of one or more latency promoting genes wherein the virus genotype comprises a wild-type genome with an alteration consisting essentially of an alteration of one or more of:
a latency promoting gene whose sequence is at least about 90% identical to UL138;
a latency promoting gene whose sequence is at least about 90% identical to UL140;
a latency promoting gene whose sequence is at least about 90% identical to UL141;
a latency promoting gene whose sequence is at least about 90% identical to UL142;
UL138;
UL140;
UL141;
or UL142.

23. The human cytomegalovirus of claim 22, further comprising a phenotype of having reduced pathogenicity, wherein such reduced pathogenicity results from attenuation of lytic replication of the virus.
